(19) United States
(12) Patent Application Publication
(10) Pub. No.: US 2022/0332742 A1
(43) Pub. Date: Oct. 20, 2022

(54) Naringin-Loaded Metal Organic Frameworks

(71) Applicant: Colgate-Palmolive Company, New York, NY (US)

(72) Inventors: Viktor DUBOVOY, Cresskill, NJ (US); Jane ONG, Franklin Park, NJ (US); James MASTERS, Ringoes, NJ (US); Long PAN, Somerset, NJ (US)

(73) Assignee: Colgate-Palmolive Company, New York, NY (US)

(21) Appl. No.: 17/723,953

(22) Filed: Apr. 19, 2022

Related U.S. Application Data
(60) Provisional application No. 63/177,197, filed on Apr. 20, 2021.

(51) Int. Cl. C07F 15/02; A61K 31/7048; A61K 8/19; A61K 8/60; A61Q 11/00

(52) U.S. Cl. CPC C07F 15/025; A61K 31/7048; A61K 8/19; A61K 8/602; A61Q 11/00; A61K 2800/92

(57) ABSTRACT

This invention relates to compositions, including oral care compositions, comprising naringin-loaded metal-organic frameworks (MOFs), such as naringin-loaded MIL-101(Fe), as well as methods of using and of making these compositions.

### BACKGROUND

This invention relates to compositions, including oral care compositions, comprising naringin-loaded metal-organic frameworks (MOFs), such as naringin-loaded MIL-101(Fe), as well as methods of using and of making these compositions.

Naringin is a flavanone glycoside, commonly found in grapefruits and citrus fruits. It has been documented to exhibit health benefits on diverse applications, such as antimicrobial activities, wound healing, periodontal diseases, diabetes mellitus and rheumatological disorders. Furthermore, it was demonstrated to inhibit the growth of periodontal pathogens as well as some common oral microorganisms in vitro. Naringin has also been suggested as a potential immune system promotor and as a possible anti-neoplastic agent, among other potential therapeutic uses. See, e.g., Jiang, et al., Frontiers Pharmacol. 12:637782 (2021), Bharti, et al., Planta. Med. 80(6):437-51 (2014), Salehi, et al., Pharmaceuticals (Basel) 12(1) (2019), Adamczek, et al., J. Clin. Med. 9(1) (2019).

Naringin has been promoted for the treatment of diverse diseases, including asthma, hyperlipidemia, diabetes, cancer, and hyperthyroidism. See, e.g., Chen, et al., Pharm. Biol. 54(12):3203-3210 (2016). However, its therapeutic efficacy is limited by its susceptibility to degradation due to pH, its ease of oxidation, and its poor solubility in aqueous media.

Tooth demineralization is a chemical process by which minerals, mainly calcium, are removed from any of the hard tissues, i.e., enamel, dentine, and cementum. The effects of demineralization may be reversed if there is sufficient time to allow remineralization to occur to counteract the acids in the oral cavity. Remineralization is beneficial for the aging population who experience gum recession as well as patients with severe periodontitis with obvious root exposure. Remineralization may further offer protection against cavity progression. A remineralization effect of flavonoids, including naringin, on artificial root caries is reported; however; the flavonoids showed to be less effective than fluoride.

Matrix metalloproteinases (MMPs) have been suggested to play an important role in the destruction of dentine organic matrix following demineralization by bacterial acids. Increasing zinc concentration has been shown to inhibit dentine-MMP dependent collagen degradation.

Toothpaste compositions comprising naringin admixed with other metal salts, such as zinc citrate, are known, as are toothpaste composition comprising naringin-metal complexes. See, e.g., U.S. Pat. No. 10,548,829. However, there remains a need for improved oral care compositions with a better efficiency for delivering naringin to the tissues of the oral cavity. It is further desirable to provide compositions which are capable of slow, steady delivery of naringin over time, to minimize any side effects.

Considerable attention recently has been focused on the hybrid porous solids known as metal-organic frameworks (MOFs) given their tunable structure and controllable porosity, which therefore are suited to serve as a nano-carrier for naringin delivery. In addition, some MOFs can be biodegradable due to the presence of relatively labile metal ligand bonds, leading to rapidly degrade the composite materials. A non-toxic porous iron (III)-based-organic framework, known as MIL-101(Fe), has emerged as a leading candidate for the delivery of poorly water-soluble pharmacological substances. It has a large loading capacity, high porosity, and mixed hydrophobic and hydrophilic properties, and these make it an ideal candidate for naringin controlled release. MIL-101(Fe) possesses two meso-cages corresponding to two windows which are ideally sized to allow naringin to easily diffuse into the pores and reside within the mesocages. Moreover, the hydrophobic interaction between naringin and the MOF structure can make it possible for controlled release.

### BRIEF SUMMARY

The present disclosure provides compositions, including oral care compositions comprising naringin-loaded metal organic frameworks (MOFs). In some embodiments, the MOF is MIL-101(Fe). In some embodiments, the composition provides slow, controlled release of the naringin into the body (e.g., the oral cavity). The present disclosure further provides methods of loading naringin into MOFs, such as MIL-101(Fe).

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

Metal-organic frameworks are coordination networks composed of metal ions and organic ligands which bridge the metal ion centers. The frameworks form roughly spherical clusters containing many ligand molecules and many metal centers. The clusters have pores of varying sizes which permit the reversible loading of other ions or molecules within the clusters in a non-covalent fashion, and generally in a non-coordinated fashion (i.e., the encapsulated material does not form a coordination complex with the MOF shell surrounding it).

In a first aspect, the present disclosure provides a compound (Compound 1) which is a naringin-loaded metal-organic frameworks (MOFs).

For example, the disclosure provides embodiments of Compound 1 as follows:
1.1 Compound 1, wherein the MOF is a transition-metal based MOF (e.g., Fe, Ti, Cr, Cu, Ni, Zn) comprising the transitional metal ions in coordination with organic ligand molecules.
1.2 Compound 1.1, wherein the organic ligand is selected from terephthalic acid (1,4-benzenedicarboxylic acid), biphenyl-4,4′-dicarboxylic acid, and trimesic acid.
1.3 Compound 1.1 or 1.2, wherein the transition metal is iron (Fe).
1.4 Compound 1.2, wherein the MOF is Mil-53(Fe), MIL-68(Fe), MIL-88(Fe), MIL-100(Fe), or MIL-101(Fe).
1.5 Compound 1.3, wherein the MOF is MIL-101(Fe), a terephthalic acid-bridged, oxo-centered, trinuclear Fe³⁺ complex.
1.6 Any of Compounds 1.1-1.5, wherein the MOF is not post-synthetically modified by ligand exchange.
1.7 Any of Compounds 1.1-1.6, wherein the MOF has a pore size from 6 to 15 Angstroms and/or a complex size of 25-40 Angstroms.
1.8 Any of Compounds 1.1-1.7, wherein the MOF is crystallized, e.g., in the form of octahedral crystals having an average diameter of 0.1 to 20 μM, e.g., 0.1 to 10 μM, 0.5 to 5 μM, 0.5 to 2 μM, or about 1 μM.
1.9 Any of Compounds 1.1-1.8, wherein the MOF is prepared using a hydrothermal technique.
1.10 Any of Compounds 1.1-1.9, wherein the naringin is reversibly loaded into the MOF.
1.11 Any of Compounds 1.1-1.10, wherein the naringin is spontaneously released from the MOF when suspended in aqueous solution in a sustained manner, e.g., over a period of at least 12 hours, or at least 24 hours, or at least 36 hours, or at least 48 hours, or at least 72 hours, or at least 96 hours, or at least 7 days, or at least 13 days, or at least 21 days, or at least 35 days, or at least 60 days.
1.12 Any of Compounds 1.1-1.11, wherein the naringin is spontaneously released from the MOF when suspended in aqueous solution to the extent of about 5% after 3 hours, or of about 8% after 26 hours, or of about 16% after 38 hours, or about 11% after 3 days, or a combination thereof.
1.13 Any of Compounds 1.1-1.11, wherein the naringin is spontaneously released from the MOF when suspended in aqueous solution to the extent of not more than 5% after 3 hours, or not more than 10% after 24 hours, or not more than 20% after 38 hours, or not more than 10% after 3 days, or a combination thereof.
1.14 Any of Compounds 1.1-1.13, wherein the naringin is loaded into the MOF in an amount of 10-20 weight % of the total weight of the naringin-loaded MOF, e.g., 10-15 weight %.
1.15 Any of Compounds 1.1-1.14, wherein two molecules of naringin are loaded into each octahedral unit of the MOF.
1.16 An oral care composition comprising Compound 1 or any of 1.1-1.15, in admixture with an orally acceptable carrier or base, and one or more orally acceptable excipients.
1.17 Composition 1.16, wherein the composition has a pH of between 5 and 9, or a pH between 6 and 8, or a pH between 6.5 and 7.5, or a pH between 6.9 and 7.1, or a pH of about 7.
1.18 Any foregoing composition, further comprising a zinc ion source, e.g., selected from zinc chloride, zinc fluoride, zinc citrate, zinc lactate, zinc acetate, zinc glycinate, zinc phosphate, zinc oxide, zinc pyrophosphate, and zinc hydroxide, wherein said zinc is not complexed to said naringin, optionally wherein said zinc ion source is present in the composition in an amount of 0.1 to 10% by weight of the composition.
1.19 Any foregoing composition, further comprising a stannous ion source, e.g., selected from stannous fluoride, stannous chloride, stannous gluconate, stannous phosphate, and stannous pyrophosphate, optionally wherein said stannous ion source is present in the composition in an amount of 0.1 to 10% by weight of the composition.
1.20 Any foregoing composition, further comprising a fluoride ion source, e.g., selected from stannous fluoride, sodium fluoride, potassium fluoride, sodium monofluorophosphate, sodium fluorosilicate, ammonium fluorosilicate, amine fluoride, ammonium fluoride, optionally wherein said fluoride ion source is present in the composition in an amount of 0.1 to 10% by weight of the composition
1.21 Any foregoing composition, further comprising a phosphate salt, e.g., an alkali metal or alkaline earth metal (e.g., Na, K, Ca, Mg) orthophosphate, pyrophosphate, tripolyphosphate, tetraphosphate, hexaphosphate, or hexametaphosphate, or combination thereof, optionally wherein said phosphate salt or mixture thereof is present in the composition in an amount of 0.1 to 10% by weight of the composition.
1.22 Composition 1.21, wherein the phosphate salt is selected from a sodium orthophosphate, a potassium orthophosphate, sodium pyrophosphate, potassium pyrophosphate, sodium tripolyphosphate, potassium tripolyphosphate, sodium tetraphosphate, potassium tetraphosphate, sodium hexametaphosphate and potassium hexametaphosphate.
1.23 Any foregoing composition, further comprising a buffer such as an acid or base or conjugate acid-base pair, e.g., one or more of sodium hydroxide, potassium hydroxide, hydrochloric acid, phosphoric acid, citric acid, lactic acid, malic acid, an alkali metal citrate, an alkali metal lactate, an alkali metal malate, an alkali metal orthophosphate, optionally wherein said zinc ion source is present in the composition in an amount of 0.1 to 10% by weight of the composition
1.24 Any foregoing composition, further comprising an anionic surfactant, e.g., sodium lauryl sulfate or sodium lauryl ether sulfate, optionally wherein said anionic surfactant is present in the composition in an amount of 0.1 to 10% by weight of the composition.
1.25 Any foregoing composition, further comprising a zwitterionic surfactant, e.g., cocamidopropyl betaine, optionally wherein said zwitterionic surfactant is present in the composition in an amount of 0.1 to 10% by weight of the composition,
1.26 Any foregoing composition, further comprising a nonionic surfactant, e.g., polyethylene glycols, or ethylene oxide/propylene oxide block copolymers (e.g., poloxamers), optionally wherein said nonionic surfactant is present in the composition in an amount of 0.1 to 10% by weight of the composition.
1.27 Any foregoing composition wherein the composition further comprises one or more of water, a thickener (e.g., xanthan gum or carboxymethyl cellulose, such as sodium salt), an abrasive (e.g., silica), a foaming agent, a vitamin, a humectant (e.g., glycerin, sorbitol, propylene glycol, or a mixture thereof), a sweetener, a flavorant, a pigment, a dye, an anti-caries agent, an anti-bacterial agent, a whitening agent, a desensitizing agent, a preservative, or a mixture thereof.
1.28 Any foregoing composition wherein the composition comprises a whitening agent, wherein the whitening agent is selected from hydrogen peroxide, cPVP-hydrogen peroxide complex, and potassium monopersulfate, or a mixture thereof.
1.29 Any foregoing composition wherein the composition further comprises a desensitizing agent selected from potassium chloride, strontium chloride, or a mixture thereof.
1.30 Any foregoing composition wherein the composition is a dentifrice, e.g., toothpaste tooth or a gel.
1.31 Any foregoing composition wherein the composition is a mouthwash.
1.32 Any foregoing composition wherein the composition is single phase or dual phase.
1.33 Any foregoing composition, wherein the composition comprises abrasive (e.g., silicas) in an amount of 1-30% by weight of the composition, e.g., 10-30%, or 20-25%, or 15-20%.
1.34 Any foregoing composition, wherein the composition releases naringin in a sustained manner to the tissues of the oral cavity, e.g., over a period of at least 12 hours, or at least 24 hours, or at least 36 hours, or at least 48 hours, or at least 72 hours, or at least 96 hours
1.35 Any of the foregoing compositions, wherein the composition is effective upon application to the oral cavity, e.g., by rinsing and/or brushing, to treat or prevent gingivitis, plaque, dental caries, dental enamel erosion, gum recession, and/or dentinal hypersensitivity.
1.36 A pharmaceutical composition comprising Compound 1 or any of 1.1-1.15, in admixture with a pharmaceutically acceptable carrier or base, and one or more pharmaceutically acceptable excipients.

In another aspect, the present disclosure provides a method of treatment or prevention of gingivitis, plaque, dental caries, dental enamel erosion, gum recession, and/or dentinal hypersensitivity, the method comprising the application to the oral cavity of a person in need thereof, of an oral care composition (e.g., a dentifrice or mouthwash) as described herein, e.g., by rinsing and/or brushing, for example, one or more times per day. In another aspect, the present disclosure provides a method of killing oral bacteria and/or improving oral immune cell function (e.g., T cell function), the method comprising the application to the oral cavity of a person in need thereof, of an oral care composition (e.g., a dentifrice or mouthwash) as described herein, e.g., by rinsing and/or brushing, for example, one or more times per day.

Alternatively, the present disclosure provides a Composition according to the present disclosure for use in the treatment or prevention of gingivitis, plaque, dental caries, and/or dental hypersensitivity. Alternatively, the present disclosure provides a Composition according to the present disclosure for use in killing oral bacteria and/or improving oral immune cell function (e.g., T cell function).

The methods of this aspect may comprise applying any of the compositions as described herein to the teeth, e.g., by brushing or rinsing, or otherwise administering the compositions to the oral cavity of a subject in need thereof. The compositions can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

In another aspect, the present disclosure provides a method of treatment or prevention of a disease selected from cancer, bacterial infection (e.g., Gram-positive infection and/or Gram-negative infection), asthma, hyperlipidemia, diabetes, cancer, and hyperthyroidism, the method comprising administering to a patient in need thereof, of a pharmaceutical composition comprising naringin-loaded MOF, as described herein. A pharmaceutical composition comprises the naringin-loaded MOF admixed with at least one pharmaceutically acceptable excipient or diluent, and may include compositions for oral (i.e., enteral), nasal, pulmonary, topical, ophthalmological, or parenteral administration (e.g., intravenous, intramuscular, or subcutaneous injection).

As used herein, an “oral care composition” refers to a composition for which the intended use includes oral care, oral hygiene, and/or oral appearance, or for which the intended method of use comprises administration to the oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans.

As used herein, “anionic surfactant” means those surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C8-C22 alkyl, or acyl group. Such surfactants are employed in the form of water-soluble salts and the salt-forming cation usually is selected from sodium, potassium, ammonium, magnesium and mono-, di- or tri-C2-C3 alkanolammonium, with the sodium, magnesium and ammonium cations again being the usual ones chosen. Some examples of suitable anionic surfactants include, but are not limited to, the sodium, potassium, ammonium, and ethanolammonium salts of linear C8-C18 alkyl ether sulfates, ether sulfates, and salts thereof. Suitable anionic ether sulfates have the formula R(OC₂H₄)ₙ OSO₃M wherein n is 1 to 12, or 1 to 5, and R is an alkyl, alkylaryl, acyl, or alkenyl group having 8 to 18 carbon atoms, for example, an alkyl group of C12-C14 or C12-C16, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium laureth (2 EO) sulfate. Some preferred exemplary anionic surfactants that may be used in the compositions of the present disclosure include sodium laurel ether sulfate (SLES), sodium lauryl sulfate, and ammonium lauryl sulfate. In certain embodiments, the anionic surfactant is present in an amount of 0.01 to 5.0%, 0.1 to 2.0%, 0.2 to 0.4%, or about 0.33%.

As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C12-16) glucoside), long chain tertiary amine oxides, long chain tertiary phosphine oxides, long chain dialkyl sulfoxides, and mixtures of such materials.

In some embodiments, the nonionic surfactant comprises amine oxides, fatty acid amides, ethoxylated fatty alcohols, block copolymers of polyethylene glycol and polypropylene glycol, glycerol alkyl esters, polyoxyethylene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments, the nonionic surfactant is present in an amount of 0.01 to 5.0%, 0.1 to 2.0%, 0.1 to 0.6%, 0.2 to 0.4%, about 0.2%, or about 0.5%.

Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. Sorbitol is commonly supplied commercially as a mixture having about 30 wt. % water (i.e., 70% aqueous sorbitol). As described here in reference to quantity, the water content of the sorbitol is excluded (for example, a formulation having 47 weight % sorbitol can be made by using about 68% by weight of 70% aqueous sorbitol). In some embodiments, humectants are present at levels of 5% to 70%, e.g., 15% to 40% by weight.

The compositions of the disclosure may include abrasives. Examples of suitable abrasives include silica abrasives, such as standard cleaning silicas, high cleaning silicas, synthetic abrasive silica or any other suitable abrasive silicas. Additional examples of abrasives that can be used in addition to or in place of the silica abrasives include phosphate abrasives, for example, a calcium phosphate abrasive, e.g., tricalcium phosphate (Ca3(PO4)2), hydroxyapatite (Ca10(PO4)6(OH)2), or dicalcium phosphate dihydrate (CaHPO4.2H2O, also sometimes referred to herein as DiCal), calcium pyrophosphate, sodium metaphosphate, or potassium metaphosphate; calcium carbonate abrasive; or abrasives such as anhydrous alumina trihydrate, aluminum silicate, calcined alumina, bentonite or other siliceous materials, or combinations thereof. The abrasive is generally present in the compositions of the present invention at a concentration of about 10 to about 40% by weight and preferably about 15 to about 20 or about 30% by weight.

In some embodiments, the oral compositions further comprise polymers to adjust the viscosity of the formulation or enhance the solubility of other ingredients. Such polymers include polyethylene glycols, polysaccharides (e.g., cellulose derivatives, for example carboxymethyl cellulose (CMC), microcrystalline cellulose or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. In one embodiment, the oral care composition may contain polyvinyl pyrrolidone (PVP). PVP generally refers to a polymer containing vinylpyrrolidone (also referred to as N-vinylpyrrolidone, N-vinyl-2-pyrrolidione and N-vinyl-2-pyrrolidinone) as a monomeric unit.

Flavorings for use in the present invention may include extracts or oils from flavorful plants such as peppermint, spearmint, cinnamon, wintergreen, and combinations thereof, cooling agents such as menthol, methyl salicylate, and commercially available products such as OptaCool® from Symrise, as well as sweeteners, which may include polyols (which also function as humectants), saccharin, acesulfame, aspartame, neotame, stevia and sucralose.

Other ingredients which may optionally be included in compositions according to the present invention include other stannous salts (e.g., stannous phosphate or stannous pyrophosphate), hyaluronic acid, green tea, ginger, sea salt, coconut oil, turmeric, white turmeric (white curcumin), grape seed oil, ginseng, monk fruit, vitamin E, basil, chamomile, pomegranate, and aloe vera. Any of such ingredients may be present in an amount from 0.01% to 2% by weight of the composition, e.g., 0.01 to 1%, or 0.01 to 0.5%, or 0.01 to 0.1%.

Water employed in the preparation of oral compositions according to the present disclosure can be deionized (sometimes referred to as demineralized water) and/or free of organic impurities. As used herein, the amount of water in the compositions includes the free water which is added plus that amount which is introduced with other materials. The compositions may comprise water in amount from 10 to 40% by weight, e.g., from 10-30%, or 10-20%, or 10-15%; (e.g., 12.7% by wt.) by weight of the oral care composition.

### Examples

### Example 1—Preparation and Characterization of the Naringin-Loaded MOF

MIL-101(Fe) is prepared according to the literature procedure (hydrothermal method) with slight modification. See Bauer, S., et al., Inorg. Chem. 2008, 47 (17), 7568-76 the contents of which are incorporated herein by reference.

Briefly, 166.13 mg of the linker terephthalic acid (BDC) and 675 mg of iron (III) chloride hexahydrate are dissolved in 15 ml dimethyl formamide (DMF) in a vial and the mixture is sonicated until the solid is completely dissolved. Then the solution is transferred to an autoclave and is heated in an isothermal oven at 120° C. for 30 hours to dehydrate the product. The resulting MIL-101(Fe) product is washed with DMF and dry acetone for several days, and then dried under vacuum.

Scanning electron microscopy (SEM) is performed on a Hitachi 5800 scanning electron microscope. The product exhibits regular octahedral crystals with an average diameter of about 1 μM.

### Example 1(a): MIL-101(Fe) Loaded with Naringin at 0.5 mg/mL in 1:4 Ethanol/Water

8.6 mg of MIL-101(Fe) is dispersed into 3 mL of a solution of naringin in water/ethanol (0.5 mg mL⁻¹ of naringin, at a ratio of 1:4 v/v ethanol/water). After stirring at room temperature for 1 hour, the naringin-loaded MIL-101(Fe) is collected by centrifugation and washed several times with ethanol solution until no naringin can be detected in the supernatant solution. The resulting product, a composite powder, is then dried under vacuum.

### Example 1(c): Evaluation of Alternative Loading Conditions

To evaluate the drug loading capacity, naringin is extracted from the naringin-loaded MIL-101(Fe). 40% HF aqueous solution is used to disassemble the composite structure and methanol is used to dilute the sample (since the linker terephthalic acid is not soluble in methanol solution). The solution is then tested by UV-Vis spectra at 284 nm on a JASCO V-670 spectrometer for the quantification of naringin present. Several solutions of naringin in water/ethanol are used as standards (0.025, 0.0125, 0.005, 0.0025 and 0.00125 mg/ml).

To better understand the naringin loading process, a range of concentrations of naringin is used to determine the highest loading efficiency and loading capacity using either absolute ethanol or aqueous ethanol as the loading solvent. Loading efficiency is calculated as: (total naringin added—free non-entrapped naringin) divided by the total naringin added. Loading capacity is calculated as the amount of naringin loaded per unit weight of MIL-101(Fe), indicating the percentage of mass of the loaded MIL-101(Fe) that is due to the encapsulated naringin. The results are shown in the table below.

| | Ethanol/Water Solution | | | | Ethanol Solution | | |
|---|---|---|---|---|---|---|---|
| EtOH:H₂O Ratio | 1:2.2 | 1:4 | 1:4 | 1:4 | | | |
| Naringin Conc. (mg/mL) | 1 | 1 | 0.5 | 0.5 | 1 | 0.5 | 0.5 |
| MOF mass:solution vol. Ratio (mg/mL) | 1:0.8 | 1:0.67 | 1:0.73 | 1:0.35 | 1:1.25 | 1:0.8 | 1:0.35 |
| Loading Efficiency | 14.7% | 18.4% | 47.3% | 91.6% | 9.1% | 26.6% | 50.5% |
| Loading Capacity | 13.0% | 12.4% | 17.3% | 16.2% | 10.3% | 10.6% | 8.9% |

As shown in table, when water is added to the loading solution, the loading efficiency and capacity is significantly increased. This is likely due to the hydrophobicity of both the MIL-101(Fe) and the naringin. However, due to the limit of the aqueous solubility of naringin, the highest water content that can be used is 80% v/v (1:4 ratio ethanol:water). It is found that the highest loading efficiency, 91.61%, is achieved using 0.5 mg/mL naringin in 1:4 ethanol/water, indicating that almost all of the naringin dissolved in solution has become loaded into the MIL-101(Fe) structure. It is found that the highest loading capacity, 17.31% (17.3 mg naringin loaded into 100 mg of MIL-101(Fe), is achieved using 0.5 mg/ml naringin in 1:4 ethanol/water.

To further characterize the naringin-loaded MIL-101(Fe) product, powder X-ray diffraction (PXRD) is performed on the naringin-loaded MIL-101(Fe) of Example 1(a). Powder X-ray diffraction patterns are collected at room temperature on a Bruker D8 Advance X-ray diffractometer at 40 kV, 40 mA for Cu_{kR} (wavelength=1.5418 Angstroms), with a scan speed of 0.15 s/step and a step size of 0.05° in 2θ at room temperature. The PXRD spectrum of pure naringin shows a characteristic peak at a 20 angle of 14.8 degrees. In contrast this peak is absent from the product spectrum, indicating that naringin is not attached to the external surface of MIL-101, but rather, is encapsulated within the MOF structure.

### Example 1(d): MIL-101(Fe) Loaded with Naringin at 1 mg/mL in 1:2.2 Ethanol/Water

Naringin-loaded MIL-101(Fe) is prepared as described in Example 1(a), except using 21 mg of MIL-101(Fe) dispersed into 17 mL of 1 mg/mL naringin solution in 1:2.2 ethanol/water.

Nitrogen sorption isotherms are collected at 77 K (liquid nitrogen bath) using a Micrometrics ASAP 2020 surface area analyzer. Dry acetone is used for solvent exchange to remove the nonvolatile solvates (e.g., DMF) for about 3 days. The test samples are dried under vacuum overnight, then further dried using the “degas” function on the ASAP 2020 instrument overnight at 120° C. The BET (Brunauer-Emmett-Teller) surface area for the naringin-loaded MIL-101(Fe) is calculated to be 2927 m²/g, indicating a significant reduction in surface area compared to pristine MIL-101(Fe), which has a surface area of 3124 m²/g. Pore size distribution is also measured. The pore volume at 2.7 nm is found to be decreased from 0.22 to 0.12 cm³/g, and the total pore volume is found to be decreased from 1.51 to 1.39 cm³/g. The results show that all of the pore apertures disappear, indicating that the naringin molecules fills the pores of the MOF, thus decreasing the pore volume.

PXRD is also performed on this naringin-loaded MIL-101(Fe), and it confirms that the MIL-101 structure is intact and that the naringin is entrapped within the structure, rather than being attached to the surface.

Thermogravimetric (TGA) analysis on a TA Instruments model Q50 is performed under nitrogen from 25 to 800° C. at a speed of 10° C./minute. The results show that this naringin-loaded MIL-101(Fe) shows a mass loss of about 13% which is absent in pure MIL-101(Fe). This is consistent with the estimated naringin loading of about 13% for this batch.

Elemental analysis is also performed, and it shows that the MIL-101 material contains 33.6% carbon and 3.11% hydrogen, whereas the naringin-loaded MIL-101(Fe) contains 38.9% carbon and 3.14% hydrogen. X-ray photoelectron spectroscopy (XPS) is also performed, and this demonstrates both a shift in the oxygen 1s and carbon 1s electron peaks of about 0.75 eV, as well as a drop in the strength of the iron 2p electron signal from 4.9% in MIL-101(Fe) to 3.4% in naringin-loaded MIL-101(Fe). This increase in carbon and hydrogen and decrease in iron content is consistent with naringin loading inside the framework of the MOF, and the shift in the MPS peak suggests an electron transfer from MIL-101 to the naringin molecule.

Fourier-transform infrared spectroscopy (FTIR) is also performed, and the spectra shows that in the characteristic C(O) vibration peak at 1036 cm⁻¹ in naringin is shifted to 1044 cm⁻¹ in naringin-loaded MIL-101(Fe). This further confirms that the naringin is encapsulated in the MOF and that there is an interaction between the MIL-101(Fe) framework and the naringin molecules.

### Example 2: Release of Naringin from Naringin-Loaded MIL-101(Fe) of Example 1(a)

To evaluate the time course of release of naringin, a sample of the naringin-loaded MIL-101(Fe) from Example 1(a) is enclosed in a dialysis bag having a molecular weight cutoff of 3500. The dialysis bag is placed into 19 mL of aqueous releasing medium and stirred. At various time points, 1 mL of solution is removed, tested for the concentration of naringin by UV-Vis spectroscopy, then the sample is placed back into the medium.

It is found that at 3 hours, only 4.7% of the loaded naringin has been released into the medium. At 38 hours, only 16.5% of the load naringin has been released into the medium. When plotted against time, the data demonstrates a sustained release of naringin at a rate which is leveling off with time. The release rate at 38 hours is approximately 0.15% per hour, compared to about 1.5% per hour over the first 3 hours.

The results demonstrate that naringin loaded into the non-toxic and biodegradable MOF MIL-101(Fe) provides slow release of the active, which is expected to substantially reduce any side effects that might be associated with high dosages. Naringin is easily loaded into MIL-101(Fe) MOF in a short time with high loading efficiency, demonstrating that MOF could be an effective naringin carrier for slow delivery.

### Example 3: Release of Naringin from Naringin-Loaded MIL-101(Fe) of Example 1(d) (MIL-101(Fe) Loaded with Naringin at 1 mg/mL in 1:2.2 Ethanol/Water)

The procedure described in Example 2 is repeated using the MIL-101(Fe) loaded with naringin at 1 mg/mL in 1:2.2 ethanol/water from Example 1(d). It is found that only 7.8% of the naringin is released after 26 hours, and only 11% was released after 13 days. This suggests that the naringin loaded into MIL-101(Fe) under these conditions (1 mg/mL in 1:2.2 ethanol/water) provides ideal slow release to provide efficacy while avoiding potential adverse side effects.

This release profile is analyzed using the mathematical models of Sahlin-Peppas, Ritger-Peppas, and Higuchi. See Ebadi, A., & Rafati, A., J. Polymers & Environment 26(8):3404-11 (2018). The non-linear fitted curves generated using the three models are consistent with release of naringin controlled by the combination of Fickian diffusion and Case-II relaxation.

In order to further confirm that the released product is naringin, the naringin-loaded MIL-101(Fe) is immersed in acetone solution for one month. The resulting solution is then centrifuged, and the acetone supernatant is removed and dried under vacuum to remove the solvent. The residue is collected in d6-DMSO and analyzed by proton NMR. The resulting NMR spectrum is consistent with naringin released into the acetone solution from the MOF.

### Example 4: Antibacterial Activity of Naringin-Loaded MOF

The antibacterial activity of the prepared MOF complexes is determined against Bacillus subtilis, by calculating minimal inhibitory concentration (MIC) using the micro-dilution method. Briefly, test samples are diluted with TSB solution to 2 mg/mL, and then further serial dilutions are prepared in TSB. Bacterial cultures are incubated in TSB for 16 hours, then 100 microliter suspensions are transferred to 4 mL of fresh medium and incubated for another 6 hours in order to reach mid-log phase stage. Then, the bacteria are diluted to provide 10⁶ colony-forming units per mL (CFU/mL) based on OD₆₀₀ measurement. Bacterial cultures are treated with either naringin, MIL-101(Fe), or naringin-loaded MIL-101(Fe) prepared according to Example 1(c). The tested products are the naringin-loaded MOF prepared from 0.5 mg/ml naringin in ethanol, 0.5 mg/mL naringin in 1:4 ethanol/water solution, and 1 mg/mL naringin in 1:2.2 ethanol/water solution. After treatment, the cultures are stained 4′,6-diamino-2-phenylindole (DAPI) to show live bacteria, and with propidium iodide (PI) to show dead bacteria. The samples are then viewed under confocal microscopy for red (PI) and blue (DAPI) fluorescence. MIC is determined as the lowest concentration that completely inhibits bacterial growth. MIC value is reported in mg/mL:

| Sample | MIC (mg/mL) |
|---|---|
| Naringin | 5 |
| MIL-101(Fe) | 1.25-2.5 |
| MIL-101(Fe) loaded with naringin | 1.25-2.5 |
| using 0.5 mg/mL in ethanol | |
| MIL-101(Fe) loaded with naringin | 2.5-5 |
| using 0.5 mg/mL in 1:4 ethanol/water | |
| MIL-101(Fe) loaded with naringin | 0.6-1.25 |
| using 1 mg/mL in 1:2.2 ethanol/water | |

These results show that there is a synergistic anti-bacterial effect between the MIL-101(Fe) and the naringin encapsulated therein. MIL-101(Fe) and other MOF's are known to have some antibacterial activity. Without being bound by theory, MIL-101(Fe) may have an antibacterial effect resulting from its ability to chelate metal ions important to bacterial survival, or by causing lipid peroxidation damage to bacterial cell membranes. The results demonstrate an enhanced synergistic effect between MIL-101(Fe)'s antibacterial activity and that of the encapsulated naringin.

### Example 5: Cell Viability Effects of Naringin-Loaded MOF

The ability of naringin-loaded MIL-101(Fe) to promote the viability of EL4 T cells is evaluated using the ATP viability assay (Perkin Elmer, ATPLite 1-step Luminescence Assay). EL4 T cells are seeded at 8000 cells/mL in white 96-well plates and incubated with either naringin or naringin-loaded MIL-101(Fe) (Example 1(d)) at the following concentrations: 3.125, 6.25, 12.5, 25, 50, 100, and 200 μg/mL. The ATP kit solution is then added to each well, and a 96-well microplate reader is used to measure total luminescence. Cell viability is calculated as a percentage of the luminescence of the untreated control. The results are shown in the following table:

| | |
|---|---|
| Concentration | Cell viability (%) |

| | | |
|---|---|---|
| of Agent | Naringin | Naringin-loaded MIL-101(Fe) |

| | | | |
|---|---|---|---|
| 3.125 | mg/mL | 77.03 | 83.56 |
| 6.25 | mg/mL | 75.78 | 90.92 |
| 12.5 | mg/mL | 72.06 | 79.70 |
| 25 | mg/mL | 67.04 | 77.82 |
| 50 | mg/mL | 74.86 | 81.74 |
| 100 | mg/mL | 80.35 | 90.76 |
| 200 | mg/mL | 84.73 | 92.35 |

This data suggests that naringin-loaded MIL-101(Fe) may promote the growth of immune T cells.

### Example 6: Tumor Cell Cytotoxicity Effects of Naringin-Loaded MOF

The ability of naringin-loaded MIL-101(Fe) to kill H1299 mouse lung tumor cells is evaluated using the ATP viability assay (Perkin Elmer, ATPLite 1-step Luminescence Assay). H1299 cells are seeded at 5000 cells/mL in white 96-well plates and incubated with either MIL-101(Fe), naringin or naringin-loaded MIL-101(Fe) (Example 1(d) at the following concentrations: 0, 3.125, 25, 50, 100, and 200 μg/mL. The ATP kit solution is then added to each well, and a 96-well microplate reader is used to measure total luminescence. Cell viability is calculated as a percentage of the luminescence of the untreated control. The results are shown in the following table:

| Concentration | | Cell viability (%) | | |
|---|---|---|---|---|
| of Agent | | Naringin | MIL-101(Fe) | Naringin-loaded MIL-101(Fe) |
| 0 | mg/mL | 100 | 100 | 100 |
| 3.125 | mg/mL | 85.97 | 80.14 | 78.50 |
| 25 | mg/mL | 73.26 | 53.90 | 72.52 |
| 50 | mg/mL | 80.15 | 57.07 | 66.02 |
| 100 | mg/mL | 87.10 | 85.39 | 62.92 |
| 200 | mg/mL | 83.23 | 67.46 | 51.49 |

This data suggests that naringin-loaded MIL-101(Fe) may be useful as a cytotoxic agent to treat cancer.

### Example 7: Promotion of IL-2 and TNF-Alpha Release by Naringin-Loaded MOF

The ability of naringin-loaded MIL-101(Fe) to promote the release of IL-2 and TNF-alpha from EL4 T cells is evaluated. Enzyme-linked immunosorbent assay (ELISA) is performed according to the manufacturer specifications. Briefly, EL-4 T-cells are incubated in 96-well plates at an initial density of 8000 cells/well. The cell cultures are treated with either MIL-101(Fe), naringin or naringin-loaded MIL-101(Fe) (Example 1(d)) at the following concentrations in PBS: 0, 100, and 200 μg/mL. The plates are incubated for 24 hours at 37° C. in 5% CO₂ atmosphere. The solutions are then transferred to a V-shaped 96 well plate and centrifuged at 500 g for 5 minutes. The supernatant solutions are then collected and the recommend ELISA protocol is performed. Absorbance at 450 and 540 nm is measured using a 96-well plate reader, and the results are compared to cytokine standard solutions. The results are shown in the following table:

| Concentration | | TNF-alpha concentration (pg/mL) | | |
|---|---|---|---|---|
| of Agent | | Naringin-loaded MIL-101(Fe) | Naringin | MIL-101(Fe) |
| 0 | mg/mL | 11.46 | 11.34 | 12.58 |
| 100 | mg/mL | 9.91 | 10.64 | 12.09 |
| 200 | mg/mL | 10.68 | 9.92 | 11.05 |

| Concentration | | IL-2 concentration (pg/mL) | | |
|---|---|---|---|---|
| of Agent | | Naringin-loaded MIL-101(Fe) | Naringin | MIL-101(Fe) |
| 0 | mg/mL | 18.05 | 17.45 | 16.85 |
| 100 | mg/mL | 16.91 | 15.22 | 14.67 |
| 200 | mg/mL | 17.43 | 16.49 | 15.76 |

This data suggests that naringin-loaded MOF may be useful for promoting IL-2 and TNF-alpha release, and consequently, immune cell activation and proliferation. Naringin-loaded MOF may also be useful in improving the viability and proliferation of NK (natural killer) cells, which are the main effector cells of the innate immune response. Naringin-loaded MOF may also be useful for inhibiting pro-inflammatory effects and promoting anti-inflammatory effects in the body.

1. A compound characterized as a naringin-loaded metal organic framework (MOF).

2. The compound of claim 1, wherein the MOF is a transition-metal based MOF (e.g., Fe, Ti, Cr, Cu, Ni, Zn) comprising the transitional metal ions in coordination with organic ligand molecules.

3. The compound of claim 2, wherein the organic ligand is selected from terephthalic acid (1,4-benzenedicarboxylic acid), biphenyl-4,4′-dicarboxylic acid, and trimesic acid.

4. The compound of claim 2, wherein the transition metal is iron.

5. The compound of claim 4, wherein the MOF is Mil-53(Fe), MIL-68(Fe), MIL-88(Fe), MIL-100(Fe), or MIL-101(Fe).

6. The compound of claim 5, wherein the MOF is MIL-101(Fe), a terephthalic acid-bridged, oxo-centered, trinuclear Fe³⁺ complex.

7. The compound according to claim 1, wherein the naringin is reversibly loaded into the MOF.

8. The compound according to claim 1, wherein the naringin is spontaneously released from the MOF when suspended in aqueous solution in a sustained manner, e.g., over a period of at least 12 hours, or at least 24 hours, or at least 36 hours, or at least 48 hours, or at least 72 hours, or at least 96 hours.

9. The compound according to claim 1, wherein the naringin is loaded into the MOF in an amount of 10-20 weight % of the total weight of the naringin-loaded MOF, e.g., 10-15 weight %.

10. An oral care composition comprising the compound according to claim 1, in admixture with an orally acceptable carrier or base and one or more orally acceptable excipients.

11. The oral care composition according to claim 10, further comprising one or more of a zinc ion source, a stannous ion source, a fluoride ion source, a phosphate salt, a buffer, an anionic surfactant, a zwitterionic surfactant, or a nonionic surfactant.

12. The oral care composition of claim 10 or 11, further comprising one or more of water, a thickener, an abrasive, a foaming agent, a vitamin, a humectant, a sweetener, a flavorant, a pigment, a dye, an anti-caries agent, an anti-bacterial agent, a whitening agent, a desensitizing agent, a preservative, or a mixture thereof.

13. An oral care composition according to claim 10, wherein the composition is a dentifrice or a mouthwash.

14. A method of treatment or prevention of gingivitis, plaque, dental caries, dental enamel erosion, gum recession, and/or dentinal hypersensitivity comprising applying an oral care composition as described herein, the method comprising the application to the oral cavity of a person in need thereof, of a composition according to claim 10, e.g., by brushing or rinsing, for example, one or more times per day.

15. A method of killing oral bacteria and/or improving oral immune cell function (e.g., T cell function) comprising applying an oral care composition as described herein, the method comprising the application to the oral cavity of a person in need thereof, of a composition according to claim 10, e.g., by rinsing and/or brushing, for example, one or more times per day.
